(19) United States
(12) Patent Application Publication
(10) Pub. No.: US 2008/0221470 A1
(43) Pub. Date: Sep. 11, 2008

(54) RESPIRATORY SENSOR ADAPTERS FOR NASAL DEVICES

(76) Inventors: Elliot Sather, San Francisco, CA (US); Toru Mino, Chicago, IL (US); Arthur Ferdinand, San Jose, CA (US); Bryan Loomas, Los Gatos, CA (US); Ryan Kendall Pierce, Carl Junction, MO (US); Rajiv Doshi, Stanford, CA (US)

Correspondence Address: SHAY GLENN LLP, 2755 CAMPUS DRIVE, SUITE 210, SAN MATEO, CA, 94403, US

(21) Appl. No.: 12/044,868

(22) Filed: Mar. 7, 2008

Related U.S. Application Data
(60) Provisional application No. 60/905,850, filed on Mar. 7, 2007.

(51) Int. Cl. A61B 5/085; A61B 5/08

(52) U.S. Cl. 600/533; 600/537

(57) ABSTRACT

Described herein are nasal respiratory devices including an attachment site for a sensors and sensor adapters for securing at least a portion of a sensor to a nasal respiratory device. Sensor adapters typically have a body frame having at least two regions: an attachment region for securing the sensor adapter to the nasal respiratory device; and a sensor connector region for securing a sensor across from an outlet of the nasal respiratory device.

### CROSS REFERENCE TO RELATED APPLICATIONS

This application claims priority to U.S. Provisional Patent Application 60/905,850, titled “Nasal Devices” (filed Mar. 7, 2007), herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND OF THE INVENTION

Detection and treatment of patients suffering from breathing disorders often requires that the patent's breathing be monitored. Monitoring may be particularly important during treatment, because it allows a physician to estimate the efficacy of treatment, and may permit dynamic modification of the treatment. For example, it may be helpful to monitor respiration in patients suffering from, or at risk for, medical conditions such as snoring, sleep apnea (obstructive, central and mixed), Cheyne Stokes breathing, UARS, COPD, hypertension, asthma, GERD, heart failure, and other respiratory and sleep conditions. Sleep labs may monitor patients to diagnose these and other conditions of sleep disordered breathing. Monitoring typically involves taping a sensor to the subject or applying a mask including a sensor over the subject's nose and/or mouth.

Unfortunately, applying a sensor to a subject in this fashion may be uncomfortable, and may make it even harder for the patient to sleep, confounding the diagnosis and treatment. This may be particularly true when sensors are used in combination with treatments involving a medical device that is worn on the subject's face, nose, and/or mouth. If a separate sensor is used, it may be difficult to match the sensor to the treatment system, which may add to patient discomfort, as the monitoring device and the treatment device must both be worn concurrently. In addition to the loss of comfort, combining sensing and treatment systems may also result in a loss of accuracy, as sensing may interfere with the function of treatment systems. Such problems may persist even with currently available treatment systems that include an integrated monitoring sensor or sensors.

For example, positive-pressure devices such as PAP (e.g., CPAP) devices are widely used to treat sleep disordered breathing. PAP devices typically include a mask or nasal pillow which is held against the subject's face, and connected to a device for supplying positive pressure air. PAP devices are active devices, because they actively regulate pressure by providing positive flow. Systems including sensors to determine respiratory pressure during treatment are often complex, in part because of the difficulty in assessing breathing flow rate in the presence of active pressure.

Recently, devices and methods for treating breathing disorders using a passive airflow resistor have been developed. These devices are typically much smaller and lighter and therefore may be more comfortable. Examples of these devices may be found in U.S. patent application Ser. Nos. 11/298,640, titled “NASAL RESPIRATORY DEVICES” (filed Dec. 8, 2005); U.S. patent application Ser. No. 11/298,339, titled “RESPIRATORY DEVICES” (filed Dec. 8, 2005); U.S. patent application Ser. No. 11/298,362, titled “METHODS OF TREATING RESPIRATORY DISORDERS” (filed Dec. 8, 2005); U.S. patent application Ser. No. 11/805,496, titled “NASAL RESPIRATORY DEVICES” (filed May 22, 2007); U.S. patent application Ser. No. 11/811,339, titled “NASAL DEVICES” (filed Jun. 7, 2007); U.S. patent application Ser. No. 11/759,916, titled “LAYERED NASAL DEVICES” (filed Jun. 7, 2007); U.S. patent application Ser. No. 11/811,401, titled “NASAL RESPIRATORY DEVICES FOR POSITIVE END-EXPIRATORY PRESSURE” (filed Jun. 7, 2007); U.S. patent application Ser. No. 11/941,915, titled “ADJUSTABLE NASAL DEVICES” (filed Nov. 16, 2007); and U.S. patent application Ser. No. 11/941,913, titled “NASAL DEVICE APPLICATORS” (filed Nov. 16, 2007). Each of these references is herein incorporated by reference in its entirety.

FIGS. 1A and 1B illustrate one variation of a nasal respiratory device having a passive airflow resistor. In FIGS. 1A and 1B, the nasal respiratory device includes an airflow resistor 105 that is positioned in a central passageway through the device. The airflow resistor in this example is a flap valve device. The airflow resistor is configured so that the expiratory airflow through the passageway has a higher resistance than inspiratory airflow. For example, the flap valve 109 opens virtually completely during inspiration to allow airflow through the device, but remains closed during expiration (as shown in FIG. 1A). The flap valve is prevented from opening during expiration by two (or more) flap valve limiters 111 which at least partially span the passageway. The nasal device of FIGS. 1A and 1B also includes two leak pathways 107, 107′, which remain open even during expiration. Careful configuration of the leak pathways and airflow resistors allows the resistance and/or flow rates during inspiration and expiration to be controlled. For example, a nasal respiratory device may include a resistance to expiration that is between about 0.01 and about 0.25 cm H₂O/ml/sec and a resistance to inhalation that is between about 0.0001 and about 0.05 cm H₂O/ml/sec when the resistance is measured at 100 ml/sec.

A nasal respiratory device typically also includes a holdfast that secures the device to the nose, so that the airflow resistor is in communication with the nasal passageway. In FIGS. 1A and 1B the holdfast is an adhesive holdfast that extends from the central passageway and allows the flexible attachment of the device to the nose. Other types of holdfasts, including compressible or compliant holdfasts that at least partially insert into the nose, may also be used.

The passageway of the nasal device shown in FIG. 1A and 1B is a stiff body region that is formed from an inner body rim 117 (in FIG. 1B) and an outer body rim 115 (in FIG. 1A). Other nasal respiratory devices may not include a stiff (or semi-stiff or flexible) rim. The inner body region 117 may also act as an aligner that helps position the device in the nose.

Nasal respiratory devices such as the nasal device shown in FIGS. 1A and 1B may be used to treat a number of respiratory disorders, including sleep disordered breathing such sleep apnea and/or snoring. However, because these devices are worn over the subject's nose, monitoring breathing while wearing the device may be difficult. Thus, it would be beneficial to provide devices, systems and methods for monitoring breathing using similar devices that address the problems identified above. In particular, there is a need for systems for monitoring breathing that are accurate and non-intrusive, and are compatible with nasal respiratory devices having passive airflow resistors. Described below are sensor adapters for nasal respiratory devices having a passive airflow resistor and methods of using them, including systems for monitoring breathing when using a nasal respiratory device. These devices, systems and methods may be used to monitor treatment of a sleep disorder.

### SUMMARY OF THE INVENTION

Described herein are sensor adapters for use with a nasal respiratory device. Sensor adapters typically have a body frame having at least two regions: an attachment region for securing the sensor adapter to the nasal respiratory device; and a sensor connector region for securing a sensor detector input for a sensor in communication with one or more outlets of the nasal respiratory device. The attachment region may be referred to as an attachment site and the sensor connector region may be referred to as a sensor connector.

In general, a sensor adapter is configured to be used with passive-resistance nasal respiratory devices. Passive-resistance nasal respiratory devices typically have a passive airflow resistor (e.g., a flap valve), and may also be referred to herein as simply “nasal respiratory devices” or “nasal devices”. The sensor adapters described herein are configured so that they may be attached to the distal (external) side of a nasal respiratory device without interfering with the activity of the nasal respiratory device. In particular, the body frame of the sensor adapter is configured so that the sensor adapter does not substantially limit the airflow or otherwise alter the resistance through the nasal respiratory device. For example, the body frame may project only slightly over the distal airflow pathway openings of the nasal respiratory device when attached to the nasal respiratory device. In some variations the body frame includes openings (e.g., passages, windows, holes, etc.) that allow airflow substantially unencumbered from the distal airflow pathway openings of the nasal respiratory device.

These devices and systems may be used with a sensor for measuring a parameter of breathing such as pressure, airflow, temperature, or the like. The sensor adapters may include a connector for the sensor detector input of a sensor. A “sensor detector input” typically refers to the sampling region of a sensor. For example, a sensor detector input may be a cannula (e.g., a nasal cannula), the sensor transducer region, or a connector to the transducer region.

In some variations, the sensor adapters include a sensor. The sensor adapters described herein may also be referred to as a cannula adapter.

For example, described herein are sensor adapters configured to attach to a passive-resistance nasal respiratory device without substantially altering the resistance to airflow through the nasal respiratory device. A sensor adapter may include a body frame having a sensor connector that is configured to secure a sensor detector region of a sensor in communication with an opening on the nasal respiratory device and an attachment site configured to mate with the nasal respiratory device and secure the sensor adapter thereto without limiting airflow through the nasal respiratory device.

In some variations, the sensor adapter is configured to secure the sensor detector input of a sensor in a predetermined position with respect to the nasal respiratory device. In some variations the sensor adapter includes a sensor. For example, the sensor detector input may be attached to a sensor connector region of the body frame.

The body frame of the sensor adapter may also be referred to as the body of the sensor adapter. The body frame is configured to position the sensor detector input of a sensor (e.g., a cannula connected to a pressure sensor, or the transducer of a thermocouple/thermister) in communication with one or more opening through a nasal respiratory device. For example, the body frame may be configured to position the sensor detector input a predetermined distance from the opening. In some variations, the body frame is configured to secure at least a portion of a sensor detector input (e.g., a cannula opening), between about 1 mm and 25 mm from an opening through the nasal respiratory device. The opening may be an expiratory opening, which may also be referred to as a leak pathway. A leak pathway is typically open during expiration and inspiration, when the resistance through the device is greater than the resistance during inspiration because of the airflow resistor. In some variations the sensor detector input is positioned to be in communication with an expiratory opening (e.g., leak pathway) and a valved opening (e.g., inspiratory pathway).

In some variations, the sensor connector region of the sensor adapter includes a channel configured to seat at least a portion of a sensor. For example, the channel may be a tube or hole into which a portion of the sensor (e.g., cannula, sensor lead, etc.) can be inserted. In another example, a portion of the sensor is configured to mate over the channel, which is a tube and may include a flange configured to mate with at least a portion of the sensor.

The attachment site region of the sensor adapter may include any appropriate attachment for connection to the nasal device. For example, the attachment region may include a surface that mates with a surface of the nasal device. In some variations, the attachment region is a snap fit region configured to secure a portion of a nasal respiratory device between two or more surfaces forming the snap fit. The attachment region may be a press-fit attachment site. The attachment region may include an adhesive material, a snap, a magnet, a hook-and-latch material, and/or a screw.

As mentioned above, in some variations the sensor adapter includes at least a portion of the sensor. The sensor or a portion of the sensor may be permanently attached. Any appropriate sensor may be used, including: a pressure transducer, a strain gauge, a thermister, a thermocouple, and an IR sensor.

One variation of a sensor adapter is a sensor adapter that is configured to attach a nasal cannula to a nasal respiratory device, the adapter comprising a body frame. This sensor adapter includes: a sensor connector having a surface to which a cannula may be secured so that a distal cannula opening is held in communication with an outlet (e.g., expiratory outlet) on the nasal respiratory device, and an attachment site configured to mate with the nasal respiratory device to secure the sensor adapter to the nasal respiratory device without substantially changing the resistance to airflow through the nasal respiratory device.

The surface of the sensor connector may be a surface against which the inner diameter of a cannula may be friction fit so as to hold the position of the cannula. Alternatively, the surface of the sensor connector may include a surface against which the outer diameter of the cannula is friction fit to hold the position of the cannula.

Also described herein are systems for monitoring respiration. These systems may be configured as systems for monitoring the treatment of a sleep disorder. For example, a system for monitoring may include: a passive-resistance nasal respiratory device having an airflow resistor configured to inhibit expiration more than inspiration; a sensor adapter configured to secure a sensor detector input of a sensor in communication with an outlet of the nasal respiratory device, wherein the sensor adapter includes a sensor connector configured to secure at least a portion of a sensor in communication with an opening on the nasal respiratory device. The sensor connector of the sensor adapter may be configured to secure the sensor detector input in communication with an expiratory outlet of the nasal respiratory device, or in communication with both an expiratory outlet and a valved outlet of the nasal respiratory device.

The system may also include a sensor configured to monitor respiration through the nasal respiratory device. For example, the sensor may be selected from the group consisting of: a pressure sensor, a thermocouple, a thermister, an IR sensor, and a strain gauge.

In some variations, the system includes a nasal cannula configured to attach to the sensor adapter so that one end of the nasal cannula is in communication with the opening on the nasal respiratory device.

Also described herein are methods of monitoring respiration, including methods of monitoring a treatment. For example, methods of monitoring treatment of a sleeping disorder are described. These methods may include the steps of: securing a nasal respiratory device to a subject's nose in communication with the subject's nasal cavity without covering the subject's mouth (wherein the respiratory device includes a passive airflow resistor configured to inhibit expiration more than inspiration); attaching a sensor to the nasal respiratory device; and monitoring respiration using a sensor connected to the nasal respiratory device.

The method may also include the step of securing a sensor adapter to the nasal respiratory device. The sensor may be attached to the sensor adapter and attached to the nasal respiratory device. In some variations, the sensor adapter is attached to the nasal respiratory device after the sensor is attached to the sensor adapter. In some variations, the method includes the step of adjusting the position of the sensor detector input. For example, the position of the sensor detector input of the sensor can be adjusted by adjusting the position of the sensor within the sensor adapter.

As mentioned, the sensor detector input of a sensor may be attached to the nasal respiratory device in communication with one or more outlets of the nasal respiratory device. In some variations, the sensor detector input is positioned to communicate with both an expiratory outlet (e.g., an outlet that is open during both expiration and inspiration) and a valve outlet (that is typically closed during expiration). In some variations the sensor detector input is positioned in communication with just the expiratory outlet. For example, the sensor may be positioned opposite a leak pathway which is opened during both expiration and inspiration. The sensor may be positioned across from the expiratory outlet only, or it may be positioned across from the expiratory outlet and another outlet of the nasal device. The sensor detector input may be spaced across from the outlet (or outlets) of the nasal device by a predetermined distance (e.g., greater than 1 mm, about 2 mm, between 1-5 mm, etc.).

In some variations, the sensor detector input is positioned within an expiratory outlet of the nasal respiratory device.

A sensor may be used to measure any appropriate respiratory parameter in order to monitor a sleep disorder. Thus, the method of monitoring a sleep disorder may include the step of monitoring airflow through the nasal respiratory device. For example, the method may include the step of monitoring air pressure from airflow through the nasal respiratory device. In some variations, the method includes the step of monitoring a temperature change from airflow through the respiratory device. The step of attaching a sensor to the nasal respiratory device comprises attaching a thermister or thermocouple, an IR sensor, a strain gauge, or the like. In some variations, the method includes the step of attaching a sensor detector input (e.g., a pressure transducer) in communication with the nasal respiratory device. For example, the method may include the step of attaching a cannula in communication with a pressure transducer to the nasal respiratory device.

Any appropriate method may be used to secure the nasal respiratory device to the subject's nose so that it is in communication with the subject's nasal cavity. For example, the device may be adhesively secured to the subject's nose, or secured by at least partially inserting into the subject's nose. A compliant material (e.g., compressible foam material) may be used to secure the device to the nose by expanding the material within the nose. Other ways that the nasal respiratory device may be attached to the nose include using a snap, Velcro, van der Waals forces, vacuum, a magnet, a friction fit, a press fit, a screw, and a hook-and-loop adhesive.

The nasal respiratory device typically includes an airflow resistor that passively resists expiration more than inspiration. For example, the airflow resistor may be a flap valve, or multiple flap valves (including valves having multiple flaps).

The method may also include attaching more than one sensor to the nasal respiratory device. In some variations, a separate sensor may be used for each nostril (which may use a single nasal respiratory device or each nostril may be attached to a separate nasal respiratory device). Thus, an additional sensor (or sensors) may be used to monitor and/or measure respiration. In some variation, a sensor that is not attached to a nasal device may also be used.

Although the methods and device described herein are generally directed towards nasal devices including sensor connectors which secure sensor detector inputs that communicate with airflow from the nasal device, these device and methods may also be adapted for use with devices that cover both the nose and the mouth, or just the mouth. Any of the sensor connectors and sensing devices may be used with such devices.

Also described herein are nasal respiratory devices having integral sensor connectors that are configured to be secured in communication with a subject's nasal cavity. These devices may include: a passageway configured to communicate with the nasal cavity; an airflow resistor in communication with the passageway, wherein the airflow resistor is configured to increase the resistance to air exhaled through the passageway more than the resistance to air inhaled through the passageway; an integral sensor connector configured to secure a sensor detector input of a sensor in communication with an opening through the device; and a holdfast configured to secure the respiratory device in communication with the nasal cavity. In some variations the holdfast is an adhesive holdfast that is configured to secure the device to the subject's nose without covering the subject's mouth, and may secure the nasal device at least partly within and/or at least partially over the subject's nasal cavity. In some variations the holdfast is a compressible holdfast that is configured to secure the respiratory device within the subject's nasal cavity by expanding to fit the subject's nasal cavity. The integral sensor connector may be configured to secure a sensor detector input of a sensor in communication with a leak pathway through the device, or in communication with a leak pathway opening and an opening from a valved pathway.

In some variations, the nasal respiratory device also includes a sensor. For example, the sensor may be selected from the group consisting of: a pressure sensor, a thermocouple, a thermister, an IR sensor, and a strain gauge.

### BRIEF DESCRIPTION OF THE DRAWINGS

FIGS. 1A and 1B show bottom (external) and top (internal) perspective views of a nasal respiratory device

FIGS. 2A and 2B show distal views of two variations of a nasal respiratory device adjacent to a nasal cannula opening.

FIGS. 3A and 3B show perspective views of a sensor adapter connected to a nasal respiratory device.

FIGS. 4A and 4B show bottom and top perspective views, respectively, of a sensor adapter.

FIGS. 4C and 4D show top and bottom views, respectively, of the sensor adapter shown in FIGS. 4A and 4B.

FIG. 5A shows a bottom view of a portion of a nasal respiratory device. FIG. 5B shows a bottom view of the nasal respiratory device of FIG. 5A with a sensor adapter attached.

FIG. 5C shows a perspective view of a nasal respiratory device and a sensor adapter.

FIG. 6A shows a cross-sectional view of a sensor adaptor connected to a nasal respiratory device.

FIG. 6B shows a bottom view of a portion of a nasal respiratory device with a sensor adapter attached.

FIG. 6C shows a sensor adapter similar to the sensor adapter shown in FIGS. 6A and 6B.

FIG. 7 shows a pair of sensor adapters aligned with a nasal respiratory device and a portion of a sensor cannula.

FIGS. 8A and 8B show perspective and side views respectively of a pair of sensor adapters that are adjustably connected.

FIG. 9A shows a perspective views of a sensor adapter. FIG. 9B shows a perspective view of the sensor adapter of FIG. 9A attached to a nasal device on a subject's nose.

FIG. 9C shows a cross-sectional view through the sensor adapter of FIGS. 9A and 9B.

FIGS. 10A and 10B show side and perspective views, respectively, of a sensor adapter including a collecting surface.

FIG. 11 shows a system including a sensor adapter.

FIG. 12 illustrates a method of monitoring respiration.

FIGS. 13A and 13B show bottom views of nasal respiratory devices having integral sensor connectors.

### DETAILED DESCRIPTION OF THE INVENTION

Described herein are sensor adapters, systems including a sensor adapter, and methods of monitoring a subject's respiration using a sensor adapter. In general, these sensor adapters include a body frame having two regions: an attachment site region for securing the sensor adapter to a nasal respiratory device and a sensor connector region for securing the sensor detector input of a sensor in communication with an inspiratory and/or expiratory outlet of nasal respiratory device. The body frame of the sensor adapter is configured so that it does not interfere with the operation of the nasal respiratory device, or permit the a portion of the sensor from interfering with the operation of the nasal device. In particular, the sensor adapter body is configured so that it does not substantially limit airflow through the nasal respiratory device or otherwise affect the resistance to airflow through the nasal respiratory device. Furthermore, the sensor connector region may control the position of the sensor so that it does not interfere with the operation of the nasal device.

Also described herein are nasal respiratory devices including integral sensor connectors. These respiratory devices typically include a sensor connector on the distal (external) face of the nasal respiratory device. These integral sensor connectors may be configured as described below for the sensor connectors that are part of a sensor adapter. Integral sensor connectors are integral to a nasal respiratory device; for example, they may be formed as part of the rim body of the nasal respiratory device.

### Sensor Adapters

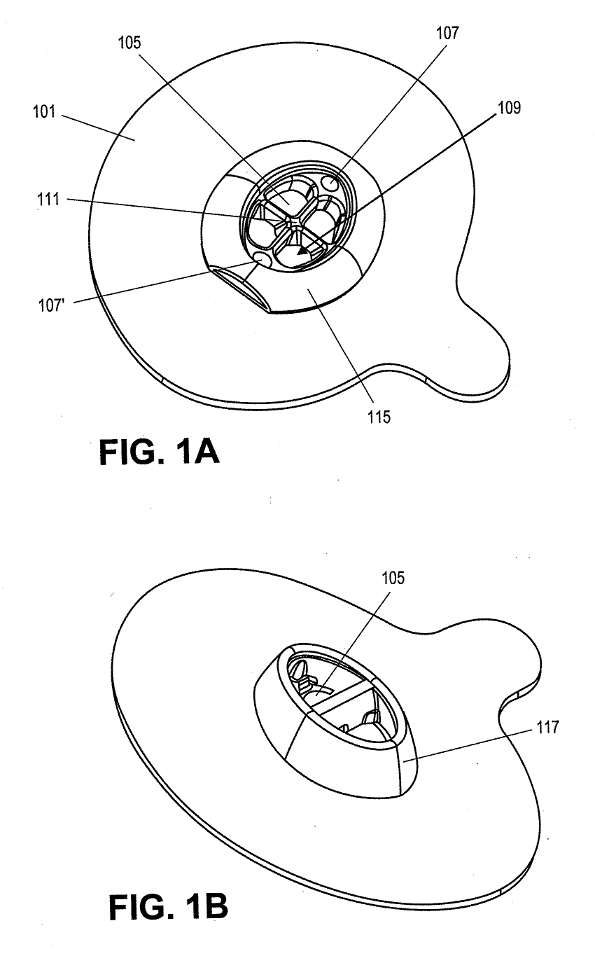

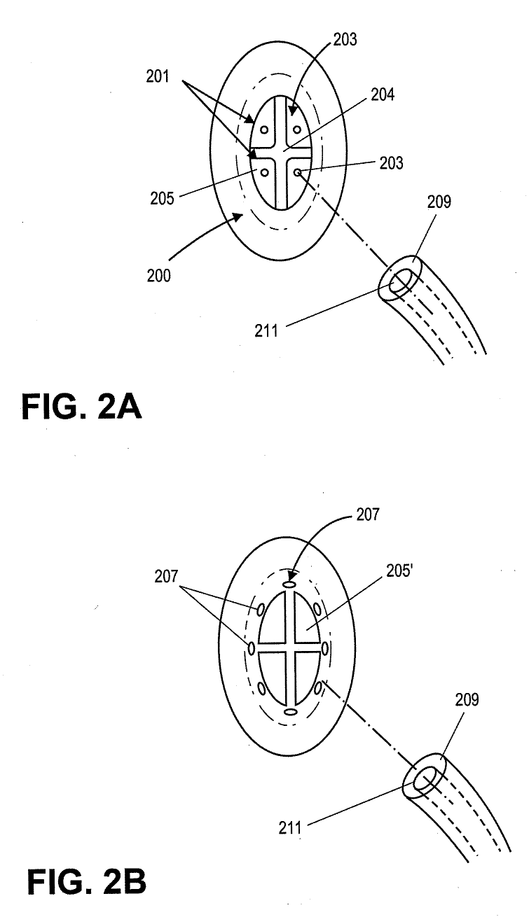

The sensor adapters described herein may be used with one or more nasal respiratory devices, particularly nasal respiratory devices that include a passive airflow resistor. An example of a nasal respiratory device is shown in FIGS. 1A and 2A, described above. Other examples may also be found in the following US patent applications, each of which was previously incorporated by reference in its entirety: U.S. Ser. No. 11/298,640, titled “Nasal Respiratory Devices”; U.S. Ser. No. 11/805,496, titled “Nasal Respiratory Devices”; U.S. Ser. No. 11/811,339, titled “Nasal Devices”; U.S. Ser. No. 11/759,916, titled “Layered Nasal Devices”; U.S. Ser. No. 11/811,401, titled “Nasal Respiratory Devices for Positive End-Expiratory Pressure”; and U.S. Ser. No. 11/941,915, titled “Adjustable Nasal Devices.”

FIGS. 2A and 2B illustrate how respiration could be monitored when using a nasal respiratory device 200. Generally, a sensor 209 may be held near or against the distal face of the nasal respiratory device to measure respiration through the nasal device. However, the position of the sensor 209 with respect to the nasal respiratory device 200 may be critical. For example, the sensor detector input of the sensor should be held secured in approximately the same region, and it should not substantially alter the function of the nasal respiratory device. Finally, the distance from an opening (or openings) through the nasal respiratory device to the sensor may be important to detecting accurate readings. A sensor adapter (not shown in FIGS. 2A and 2B) may be used to reliably secure a sensor relative to the nasal device without substantially altering the function (or resistance to expiration and inspiration) of the nasal device.

In order to get reproducible sensor readings when using a passive-resistance nasal respiratory device, it may be helpful to place the sensor in communication with more than one outlet of a nasal device. In particular, the sensor detector input may be placed in communication with an expiratory outlet (e.g., leak pathway) and a valved outlet. A valved outlet is the opening through the nasal device that is typically regulated by the airflow resistor so that it is closed (or partially closed) during expiration. Placement of the nasal device in communication with just an expiratory outlet may result in an imbalance in the magnitude of the sensor reading between inspiration and expiration, since the airflow during inspiration is typically distributed between both leak pathways and the valved openings (which are typically much larger) and during expiration the airflow is predominantly limited to the leak pathways. By positioning the sensor detector input in communication with both a leak pathway (or expiratory outlet) and valved pathway openings, the signals during both expiration and inspiration may be more balanced. In some variations the proximity of the sensor detector input to either a leak pathway and a valved pathway opening is determined by the ratio of the sizes of the opening; the sensor detector input may be closer to the smaller of the two openings, typically the leak pathway/expiratory outlet. In other variations, the sensor detector input may be further from the smaller of the two openings.

Similarly, the distance from the opening(s) and the sensor detector input of the sensor may be predetermined. If the sensor detector input is too close to an opening of the nasal respiratory device it may interfere with operation of the nasal respiratory device; if it is too far, it may not accurately sense respiration. Thus, in some variations the sensor detector input is greater than 1 mm from the nasal device outlet (e.g., leak pathway opening and/or valved opening), or greater than 2 mm away, or between 1 mm and 10 mm away.

It should be understood that when the specification refers to positioning a sensor with respect to the nasal device (e.g., in communication with an outlet of the nasal device), the region of the sensor positioned is the sensor detector input, unless the context makes clear otherwise.

It is desirable to measure respiration through the nasal device during both inspiration and expiration. A sensor can be placed in communication with one or more openings of the nasal respiratory device to measure one or more characteristic of respiration through the nasal device. As described in greater detail below, any appropriate sensor may be used, including a pressure sensor connected to a cannula, a thermister, a thermocouple, etc. A cannula 209 (connected to a pressure sensor, not shown) having an opening 211 is illustrated in FIGS. 2A and 2B.

As mentioned, the position of the sensor detector input (e.g., cannula 209) relative to the openings in the nasal device on the external side may dramatically affect the accuracy and stability of the sensor readings. For example, it may be useful to measure airflow from an expiratory opening in the nasal respiratory device. In FIGS. 2A and 2B the openings in the nasal respiratory devices are leak pathways 203, 207. In FIG. 2A the leak pathway is formed thorough the flap valve 205. In FIG. 2B the eight leak pathways are formed separately from the flap valve. In either case, this expiratory opening allows airflow during exhalation when the airflow resistor is at least partially closed.

The body frame of the sensor adapter may control the distance between a sensor (including cannula) and the external side of the nasal respiratory device. Further, the body frame of the sensor adapter is typically configured so that is does not interfere with the operation of the nasal respiratory device to which attaches. This means that the sensor adapter does not substantially limit flow through the passive nasal respiratory device to which it attaches. For example, a passive nasal respiratory device typically increases the resistance to expiration greater than the resistance to expiration, and may maintain these resistances within a predetermined range.

Returning to the exemplary passive nasal respiratory device shown in FIGS. 1A and 1B, the nasal respiratory device includes an airflow resistor 105. In this example, the airflow resistor is a flap valve 105, although any appropriate airflow resistor (e.g., ball valve, etc.) may be used. When worn by a subject, the airflow resistor increases the resistance to expiratory airflow by closing at least partially during expiration. Thus, during expiration, airflow through the device passes predominantly (or completely) through the leak pathways 107, 107′. During inspiration the airflow resistor 105 is open, and inspiratory airflow may pass through the valved opening 109 in addition to the leak pathways 107, 107′. The valved opening in this example is divided into four parts by the support struts/flap valve limiter 111. FIG. 1A shows the distal, or external side of the nasal respiratory device. When worn by a subject, the external side of the nasal device faces outward, and airflow into and out of the nasal respiratory device passes through the leak pathways 107, 107′ and the valved opening 109.

A sensor adapter typically attaches to the external side of a nasal respiratory device, such as the external side of the devices shown in FIGS. 1A, 2A and 2B. The sensor adapter body frame is configured so that it attaches on the external side of the airflow resistor so that a sensor can be secured in communication with at least a portion of an opening on the nasal respiratory device. The opening is generally an inspiratory and/or expiratory opening, such as a leak pathway 107, 107′ in FIGS. 1A and 203 and 207 in FIGS. 2A and 2B, respectively, or a valved opening 109.

The body frame of the sensor adapter is also configured so that it can attach to the nasal respiratory device without substantially altering the function (e.g., the inspiratory or expiratory resistance) of the nasal respiratory device. For example, the body frame of the sensor adapter may project only slightly over an opening of the nasal respiratory device when the sensor adapter is attached to the nasal respiratory device. Alternatively, or in addition, the body frame may include one or more openings (e.g., windows, gaps, passages, etc.) to allow airflow from the opening(s) of the nasal respiratory device to communicate with the outside environment substantially unimpeded.

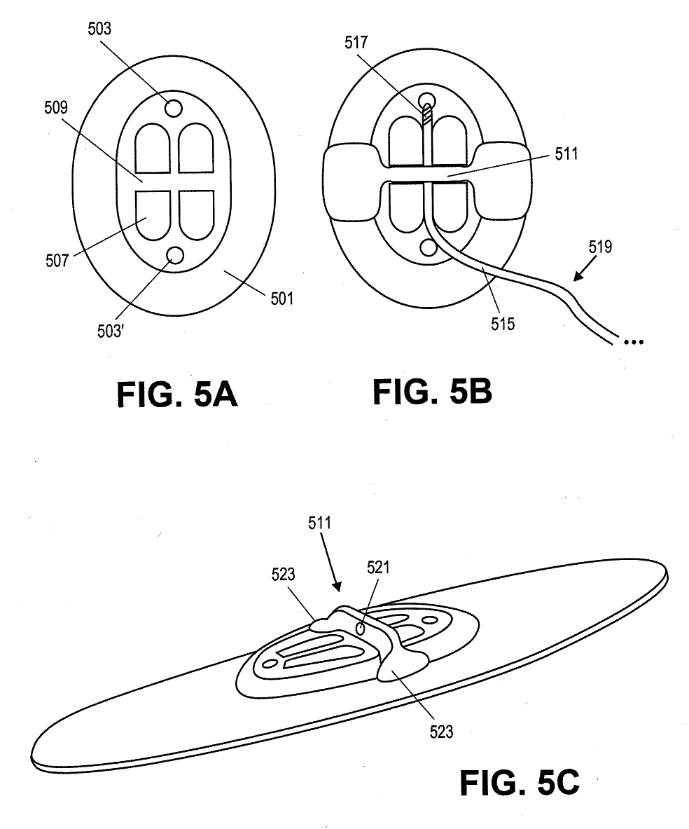

In variations of the sensor adapter that project only slightly over an opening (or openings) of the nasal respiratory device, the body frame may project over an opening of the nasal respiratory device so that it covers less than 25% (or less than 20%, less than 15%, less than 10%, less than 5%, etc.) of the openings of the nasal respiratory device. This is illustrated below in FIGS. 5A-9C.

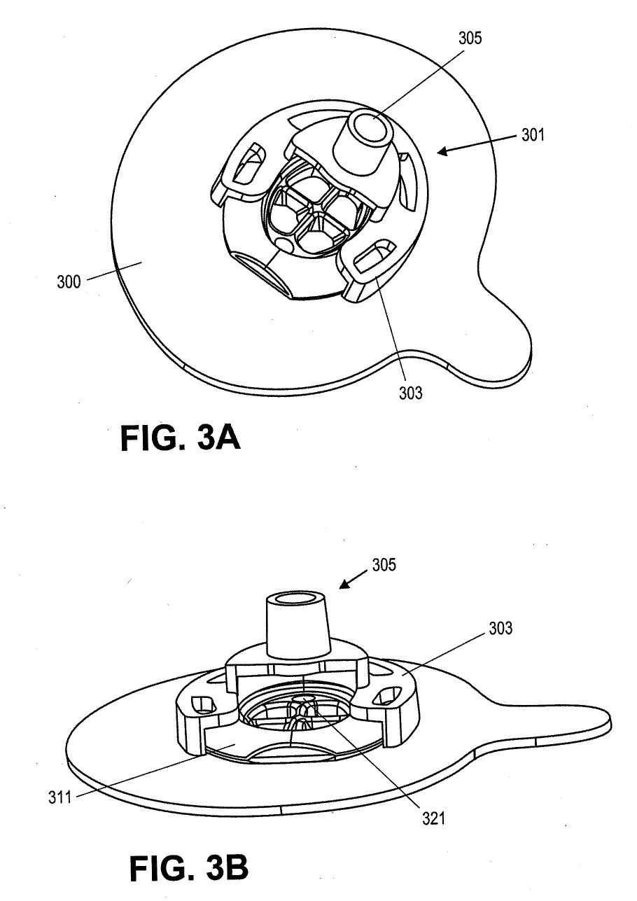

In variations in which the body frame includes one or more openings or passages, the openings or passages may be located between the attachment site region and the sensor connector. For example, the attachment site region of the body frame may be located proximally so that it contacts the distal or external face of the nasal respiratory device, and the sensor connector region may be located more distally, and the body frame may include one or more openings or windows between the distal sensor connector and the proximal attachment site. This is illustrated in more detail below in FIGS. 3A-4D and 10A-10C.

In general, the sensor adapter, including the body frame, may be made of any appropriate material or materials. Lightweight materials may be particularly preferred, as are materials appropriate for use on or near skin (e.g., biocompatible materials). Appropriate materials include may include polymers (e.g., plastics), metals (including alloys), rubbers, ceramics, wood, or the like, and combinations thereof. For example, the body frame may be made of a polymeric material such as a polypropylene, polystyrene, polyvinyl chloride, polyvinylidene chloride, polyvinyl acetate, polyacrylate, styrene-butadiene copolymer, chlorinated polyethylene, polyvinylidene fluoride, ethylene-vinyl acetate copolymer, ethylene-vinyl acetate-vinyl chloride-acrylate copolymer, ethylene-vinyl acetate-acrylate copolymer, ethylene-vinyl acetate-vinyl chloride copolymer, nylon, acrylonitrile-butadiene copolymer, polyacrylonitrile, polyvinyl chloride, polychloroprene, polybutadiene, thermoplastic polyimide, polyacetal, polyphenylene sulfide, polycarbonate, thermoplastic polyurethane, or the like. The body frame may be made at least partially of a thermoplastic resins, thermosetting resins, natural rubbers, synthetic rubbers (such as a chloroprene rubber, styrene butadiene rubber, nitrile-butadiene rubber, and ethylene-propylene-diene terpolymer copolymer, silicone rubbers, fluoride rubbers, and acrylic rubbers), elastomers (such as a soft urethane, water-blown polyurethane), and thermosetting resins (such as a hard urethane, phenolic resins, and a melamine resins).

The body frame may be made of (or coated with) a biocompatible and/or hypoallergenic material. For example, biocompatible materials that may be used include (in addition to some of those described above) biocompatible polymers and/or elastomers. Suitable biocompatible polymers may include materials such as: a homopolymer and copolymers of vinyl acetate (such as ethylene vinyl acetate copolymer and polyvinylchloride copolymers), a homopolymer and copolymers of acrylates (such as polypropylene, polymethylmethacrylate, polyethylmethacrylate, polymethacrylate, ethylene glycol dimethacrylate, ethylene dimethacrylate and hydroxymethyl methacrylate, and the like), polyvinylpyrrolidone, 2-pyrrolidone, polyacrylonitrile butadiene, polyamides, fluoropolymers (such as polytetrafluoroethylene and polyvinyl fluoride), a homopolymer and copolymers of styrene acrylonitrile, cellulose acetate, a homopolymer and copolymers of acrylonitrile butadiene styrene, polymethylpentene, polysulfones polyimides, polyisobutylene, polymethylstyrene and other similar compounds known to those skilled in the art. Teflon, Mylar, PFA, LDPE, Hytrel, HDPE and polyester may also be used.

Materials that are relatively stiff may be particularly useful for forming the sensor adapter. In addition, materials that are sterilizable may also be preferred, for example, medical grade plastics such as Acrylonitrile Butadiene Styrene (ABS), latex, polypropylene, polycarbonate, and polyetheretherketone (PEEK). The materials described above are intended as illustrations only, and other materials having similar properties may be used as well.

The attachment region of the body frame typically includes one or more attachment surfaces that are configured to secure the sensor adapter to nasal device on the distal (external) side of the nasal device. For example, the attachment surface may configured to mate with a portion of the nasal respiratory device, including the body region (forming the passageway through the nasal device), and/or the distal face of the holdfast which secures the nasal device to the subject's nose.

The attachment region may include a mechanical, chemical, magnetic, or other type of attachment to secure to the nasal respiratory device. For example, the attachment region may include an adhesive to secure to the nasal respiratory device. The attachment region may include a mechanical attachment such as a snap, screw, press-fit, or the like. The attachment region may mate with a region on the nasal device. For example, the attachment region may include a snap-fit that includes surfaces which secure a portion of the nasal respiratory device there between. In some variations the attachment region includes a hook-and-latch material (e.g., Velcro) for securing the sensor adapter to a nasal respiratory device. In some variations, the attachment region includes a magnetic material for magnetically attaching to the nasal respiratory device. The attachment region may be made of combinations of these materials.

The attachment region of the body frame may be configured to removably or permanently attach the sensor adapter to a nasal respiratory device.

The sensor connector region of the body frame generally secures at least a portion of a sensor (such as a sampling or sensor detector input of a sensor) in communication with an opening of the nasal respiratory device. The sensor connector may be configured to secure any appropriate sensor, or it may be adapted for a particular type or sensor structure.

As used herein, the term “sensor” may include any appropriate sensor for sensing and/or measuring a respiratory characteristic. The term “sensor” typically includes the sensor housing, a sensor lead, and sensor detector input or sampling region, unless specifically excluded. For example, a pressure sensor may be connected to a cannula (e.g., a hollow tube). The cannula may be considered part of the sensor (e.g., the sensor detector input of the sensor). Thus, a sensor connector may connect to a sensor (including a cannula) so that the sensor may receive input (e.g., detect information from) an opening in the nasal respiratory device. The sensor connector therefore positions the sensor detector input of the sensor in communication with an opening or openings on the external side of the nasal respiratory device.

Types of sensors that may be used include pressure sensors, a flow sensors (airflow sensors), a temperature sensors, a moisture sensors, a gas sensors (e.g., chemical sensors), or the like. Sensors may be mechanical or electronic. A sensor may include a transducer. Examples of sensor transducers include thermocouples, thermisters, strain gages, infrared sensors, or the like. A sensor may be referred to by its transducer type (i.e., “a thermister”, etc.).

In general, the sensor connector secures the sensor detector input of the sensor in communication with an opening on the nasal respiratory device by holding the sensor detector input of the sensor in communication with an opening on the nasal respiratory device, such as an expiratory opening (e.g., a leak path). In some examples the sensor detector input of the sensor is a cannula mouth. In some variations, the sensor detector input of the sensor is the transducer.

The sensor connector may be a mount that holds the sensor in position. The sensor connector may be adjustable, so that the sensor can be positioned relative to the sensor adapter and/or the nasal device. In some variations the sensor can be secured more tightly after it has been adjusted (e.g., by clamping or otherwise activating the sensor connector). A sensor connector may permanently or releasably secure a sensor.

A sensor connector may include one or more structures for holding the sensor in place. For example, the sensor connector may be configured as a surface that grips or attaches to the sensor. The sensor connector may be an opening, tube or passageway into which a portion of the sensor fits. The sensor connector may be a protrusion, tube, or prong onto which the sensor is attached. In some variations the sensor connector includes one or more adjustable surfaces that can clamp onto a portion of a sensor (e.g., sampling cannula, sensor housing, sensor lead, etc.).

The sensor connector may also be keyed to the sensor. Keying may help orient the sensor with respect to the nasal device. For example, the sensor connector may be keyed by having an opening into which the sensor inserts that is notched or flattened on one side in compliment with a projection, groove or surface of the sensor.

The sensor connector may secure the sensor in any appropriate fashion. For example, the sensor connector may be sized to friction fit to the sensor. The sensor connector may include an adhesive surface for securing the sensor. The sensor connector may include a compressible or clamping surface for securing the sensor. The sensor connector surface may interlock with the sensor (or a complimentary portion of the sensor). The sensor connector may magnetically secure the sensor within the sensor connector. The sensor connector may secure a sensor therein using an elastomeric material. For example, the sensor connector may contract around or over a portion of the sensor.

The examples below show different variations of the sensor adapters and may further illustrate variations of the sensor adapter body frame including the sensor connector region and the attachment site region.

### EXAMPLES

FIGS. 3A and 3B illustrate one variation of a system including a nasal respiratory device 300 and a sensor adapter 301 configured to secure a sensor (not shown) in communication with an opening on the distal side of the nasal respiratory device.

In FIG. 3A the sensor adapter includes a body frame that is horseshoe-shaped proximally forming the attachment site, and extends distally to form the sensor connector. Between the attachment site and the sensor connector is an opening, which is more clearly visible in FIG. 3B. The opening permits the passage of air from the opening without substantially increasing the resistance to airflow through the device.

When the nasal respiratory device shown in FIG., 3A and 3B is worn, the airflow resistor is placed in communication with the subject's nasal passage, and the adhesive holdfast secures the nasal device in place. The sensor adapter may be attached to the nasal respiratory device either before or after the device is secure to the subject's nose. For example, the nasal respiratory device maybe applied in communication with the subject's nose first. Thereafter, the sensor adapter can be attached to the nasal respiratory device. Similarly, a sensor may be attached to the system either before or after either the nasal device or sensor adapter has been applied. The sensor may be positioned within the sensor adapter.

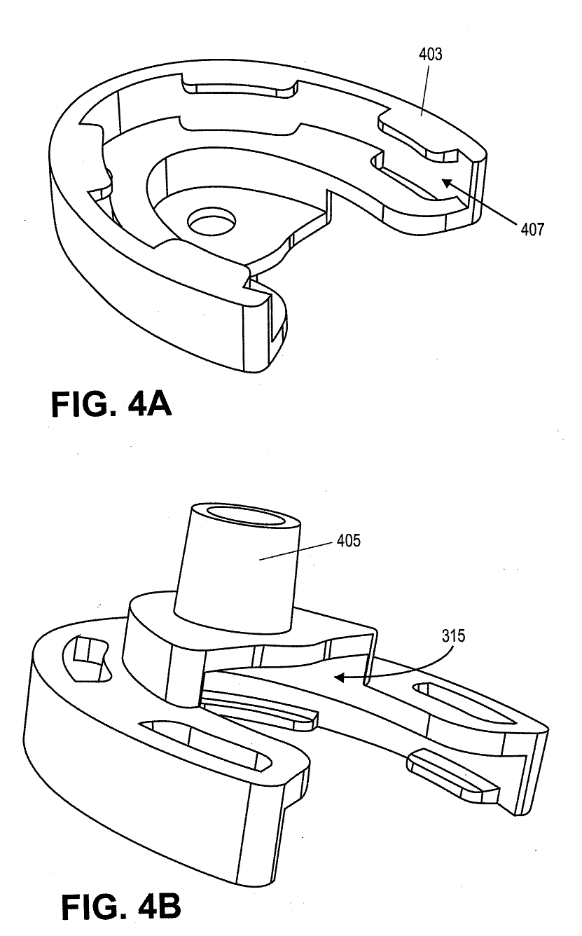

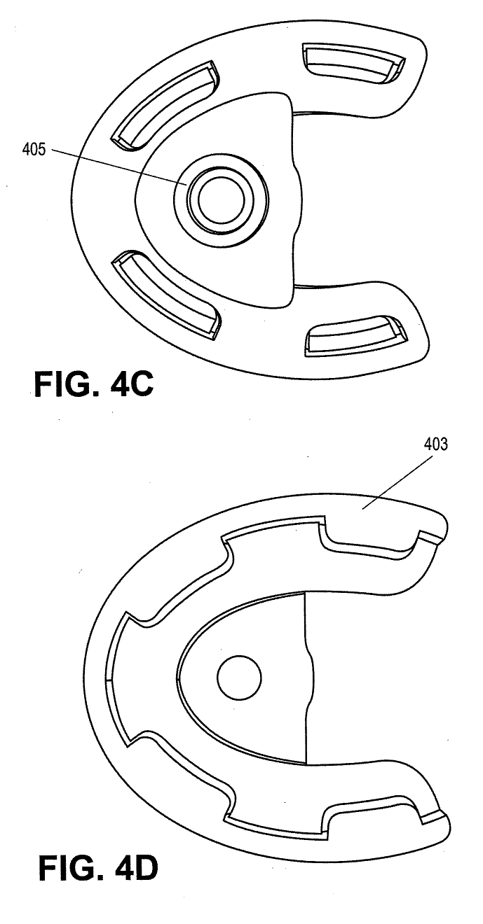

The sensor adapter shown in FIGS. 3A-4D has a snap-fit attachment site that is configured to snap onto the distal face of the nasal respiratory device 311. This attachment region 303 includes a channel (described in greater detail below) into which the external rim body region slides. The horseshoe-shaped attachment site 303 expands slightly during attachment, as the widest part of the external rim body region slide into the attachment site, and then contracts back down once the sensor adapter is secured in position. Thus, the device may be secured in place. Once the device is secured in position, the sensor connector region 305 is positioned opposite at least a portion of the expiratory opening 321 (leak pathway), which is open during expiration and inspiration in this nasal respiratory device.

Although the nasal respiratory device in FIGS. 3A and 3B is similar to the example shown in FIGS. 1A and 1B, it should be understood that any appropriate nasal respiratory device may be used. For example, the nasal respiratory device may include a compressible holdfast, and may not include a rim body region.

FIGS. 4A-4D illustrate the sensor adapter of FIGS. 3A and 3B unattached to a nasal respiratory device. FIG. 4A is a perspective view of the bottom of the sensor adapter, showing the body frame forming the attachment region 403. The attachment site region 403 includes a channel formed in an approximately “U” or horseshoe shape. As mentioned above, a portion of the nasal respiratory device may slide into this channel, to secure the sensor adapter in position. The sensor adapter may later be disengaged by sliding off of the nasal respiratory device.

FIG. 4B is another perspective view of the sensor adapter showing the distal side of the device, including the sensor connector region 405. In this variation, the sensor connector is a tube or passageway into which a sensor (e.g., cannula, sensor lead, etc.) may be placed to position it opposite an opening of the nasal device. For example, a cannula may slide into the sensor connector and be held therein by friction. Thus, the size of the opening formed through the sensor connector may be matched (e.g., approximately the same or slightly greater) to the outer diameter of the cannula. In some variations (not shown here) the sensor connector includes a stop. For example, the proximal end of the passage of the sensor connector may have a slightly smaller diameter than the distal opening into the sensor connector. This limits how far in (proximal) the sensor can be inserted. Thus, the sensor adapter may control the position of the sensor relative to the opening(s) of the nasal device.

In some variations, the sensor connector may include a washer or other seal around the inner perimeter. This seal may help secure the sensor in position and may help form a seal for reading one or more respiratory parameters.

The sensor adapter include an opening or window 315 which allows passage of air from the nasal respiratory device relatively unencumbered, while positioning the sensor connector directly over (perpendicular to) an opening on the nasal respiratory device. This roof-like structure is opened on one side, and is sufficiently large so that the sensor adapter does not provide a substantial amount of additional resistance to airflow through the nasal device. For example, the distance may be between about 1 mm and 25 mm.

FIGS. 4C and 4D show top and bottom views, respectively, of the sensor adapter shown in FIGS. 3A-4C.

FIGS. 5A-5C illustrate another variation of a system including a sensor adapter and a nasal respiratory device. FIG. 5A shows a portion of the external face of a nasal respiratory device, including the external rim body region 501. Two leak pathways 503,503′ are also visible, as is the flap-valve mechanism, including a flap valve 507 and the cross struts 509 forming the valve support and limiter. In FIG. 5B a sensor adapter 511 is attached to the rim body region 501 of the nasal respiratory device. As mentioned the sensor adapter may be attached to any appropriate portion of a nasal respiratory device, and is not limited to a rim body region.

A sensor 515 (including sensor lead 519 and sensing end 517) is secured in the sensor connector region of the sensor adapter in FIG. 5B. The sensor is held so that the sensing end 517 is positioned at least partially over the leak pathway 503. FIG. 5C illustrates another view of the sensor adapter connected to the nasal respiratory device.

In FIG. 5C it is apparent that the sensor adapter includes two attachment sites (feet 523, 523′) that attach to the rim body region 501. In this example, the attachment site may be snap-fit onto the nasal device, or they may be adhesively attached onto the nasal device. Other attachment methods may be used as well. In some variations the attachment sites align with a structure or marker on the nasal respiratory device prior to attaching. Alignment structures may help maintain a predictable orientation. Alignment structures may also help secure the sensor adapter to the nasal device.

The sensor adapter shown in FIG. 5C also includes a passageway through the body frame forming the sensor connector 521. As is illustrated in FIG. 5B, the sensor fits into the sensor connector and is held therein. In this variation, the body frame does not cover substantially extend or project over the distal airflow pathway openings. Thus, the sensor adapter does not inhibit airflow through the nasal device, and does not significantly modify resistance through the device.

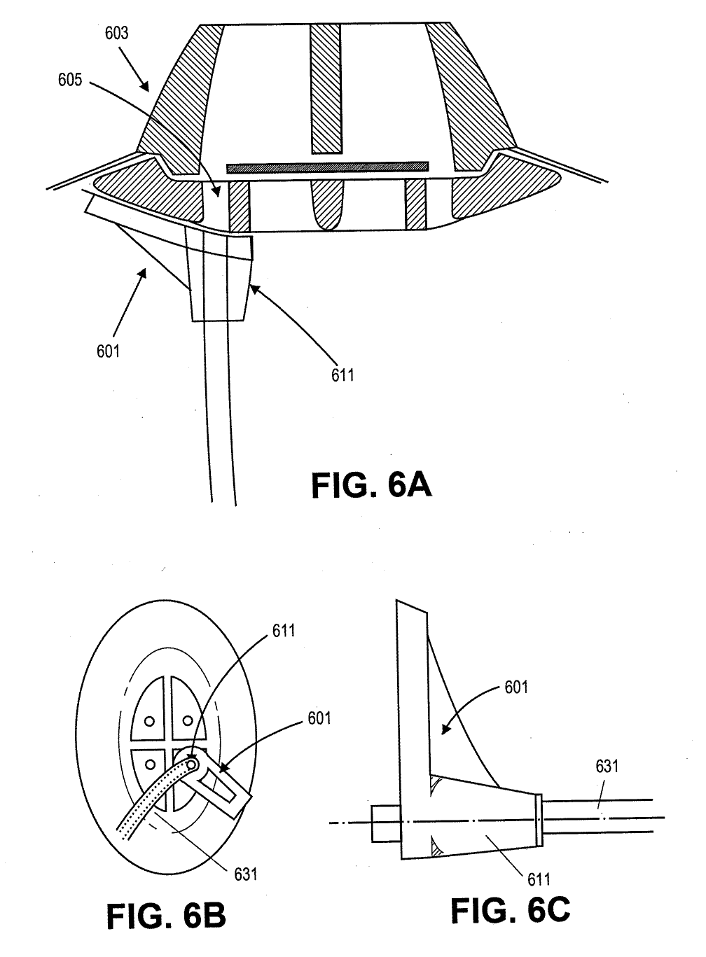

FIG. 6A shows a cross-section thorough another variation of a system for monitoring breathing including a nasal respiratory device 603 and a sensor adapter 601. In this example, the sensor adapter is configured to position the sensor detector input over the leak pathway (e.g., expiratory opening) 605 so that the sensor connector is virtually continuous with the leak pathway. In some variations, the sensor connector may project slightly into an opening of the nasal respiratory device, particularly an always-open (e.g., leak) opening. In some variations, the sensor may be secured by the sensor connector and inserted against, or partly in, an opening on the nasal respiratory device.

FIG. 6B shows a partial bottom view of a similar system (looking towards the distal end of the nasal respiratory device) in which the leak pathways are in flap valve. In this example, the sensor adapter is secured to an external face of the nasal respiratory device (e.g., the external body region), and projects only slightly over the airflow pathway opening of the nasal respiratory device. The attachment site on the sensor adapter includes a surface 609 that will contact and mate with the nasal respiratory device. In some variations, this surface includes an adhesive material so that it is secured to the nasal respiratory device. The sensor connector region of the body frame 611 includes a channel into which the sensor 631 (e.g., cannula, sensor body, etc.) may be secured.

FIG. 6C shows a sensor adapter connected to a sensor 631 (shown here as a cannula), but not yet connected to a nasal respiratory device. This sensor adapter is similar to the ones shown in FIGS. 6A and 6B, and includes a body frame having an attachment sit 609 and a sensor connector 611. As in any of the sensor adapters described herein, the sensor adapter may include additional structures or materials to secure the sensor to the sensor adapter, including a seal (e.g., washer, sealing surface, etc.), a clamp, a gasket (e.g., elastomeric band), or the like. A proximal/distal limiter may be used to control the position of the sensor relative to the opening(s) of the nasal device. For example, a proximal-distal limiter may be a notch or projection within the sensor connector.

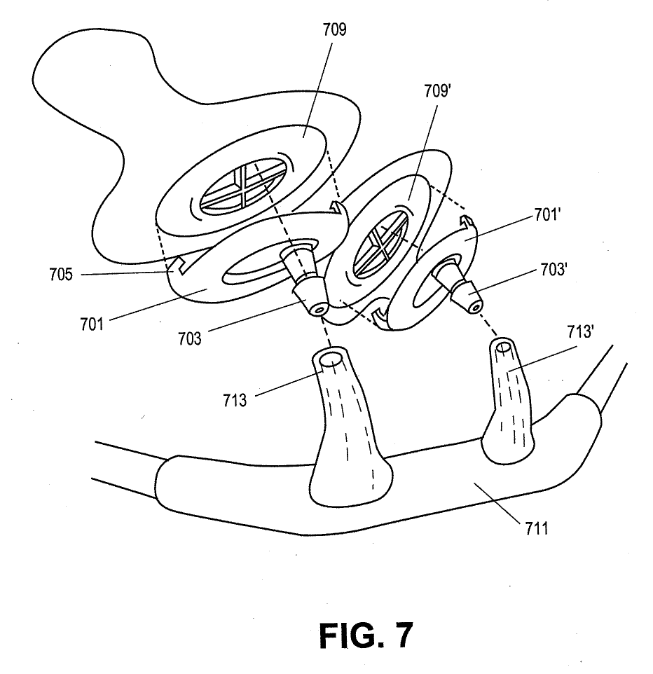

In some variations the sensor connector includes a friction flange into which a sensor can mate. FIG. 7 illustrates a pair of sensor adapters 701, 701′ that can attach to a nasal device (or devices) 709, 709′ and can then mate with a nasal cannula 711 having two prongs 713, 713′. The prongs 713, 713′ of the nasal cannula 711 can connect to the sensor connectors 703, 703′, so that the cannula prongs slide over the flange regions of the sensor connectors to mate with them.

The sensor adapters 701, 701′ shown in FIG. 7 also include annular attachments that connect to the external rim body region of the nasal respiratory device 709, 709′ via snap-fit 705. The opening of the annular attachment site allows air to flow through the nasal device substantially unimpeded. The sensor connector projects partially over an opening on the nasal respiratory device without limiting airflow through the nasal respiratory device, but still allowing sampling of air through the nasal device during respiration.

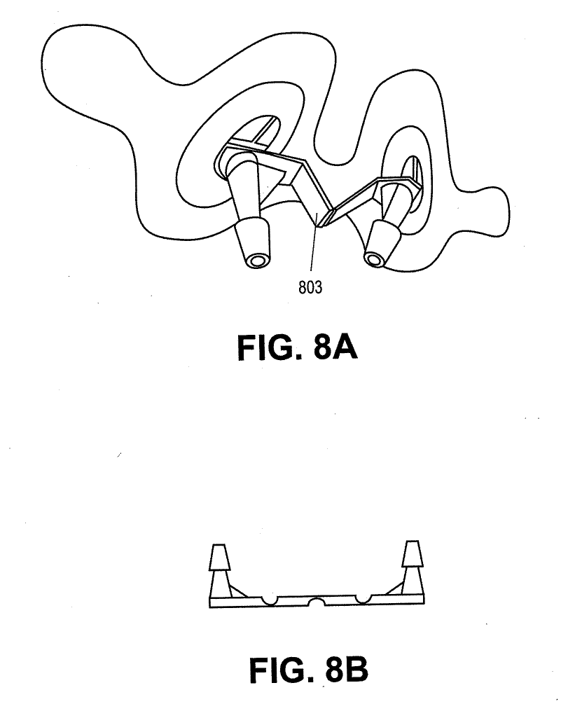

FIGS. 8A and 8B illustrate another variation of a sensor adapter that includes sensor connectors that are similar to the sensor connectors shown in FIG. 7. In this example, the pair of sensor adapters is connected by an adjustable connector 803 that permits adjustment of the spacing between the pair of sensor adapters. The attachment site regions of the sensor adapters are similar to those described above for FIGS. 6A-6C. A nasal cannula having two attachment prongs (as illustrate in FIG. 7) may be attached. FIG. 8B shows a side view of the dual-sensor adapter device shown in FIG. 8A, in the fully extended position.

The adjustable connector region 803 shown in this example is a living hinge that is made as part of the body frame. Other adjustable regions may be made using different constructions, including flexible materials (e.g., strings, fibers, etc.), bendable structures (e.g., springs, etc.), and the like.

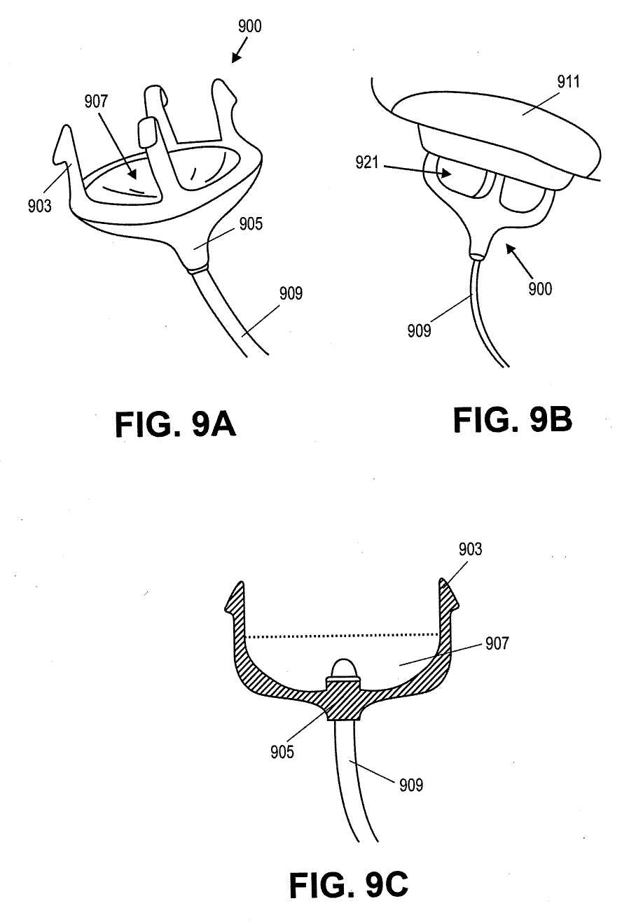

FIG. 9A-9C shows another variation of a sensor adapter that attaches to a nasal device leaving a window or gap between the attachment site and the sensor connector so that airflow through the nasal device is not inhibited. In FIG. 9A, the sensor adapter 900 in this example has an attachment site that is made up of four “legs” that can connect to a nasal device. The legs forming the attachment site(s) include a notch or flange at their ends so that they can engage a region (e.g., the rim body region) of a nasal respiratory device. When the device is attached to a nasal respiratory device (as illustrated in FIG. 9B), the body frame includes passages or windows 921, as mentioned.

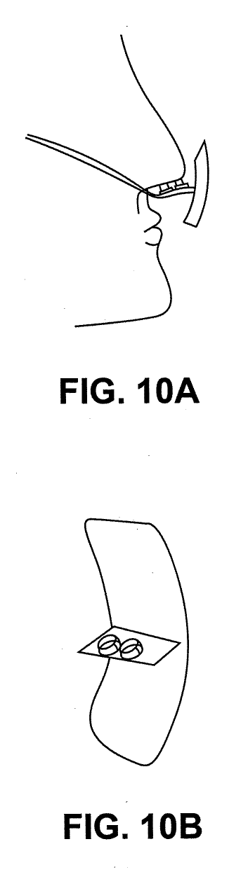

The body frame also includes a sensor connector region 905 forming a passageway into which a sensor 909 is connected. As mentioned above, the sensor may be secured in the sensor connector region by a friction fit, by an adhesive, by an elastomeric region, by a vise or clamp region, etc. In this example, the body frame also forms a collecting surface 907. The collecting surface illustrated in FIG. 9A is concave, or funnel-shaped. A collecting surface may help funnel airflow from the nasal device to the sensor, for detection. A collection surface may have different shape (e.g., it may be flat or convex), and may be of any appropriate size. In general, a collection surface may be included as part of any of the sensor adapters described herein. FIGS. 10A and 10B illustrate another variation of a collection surface that may be included as part of a sensor adapter.

FIG. 9C illustrates a cross-section through the sensor adapter shown in FIGS. 9A and 9B.

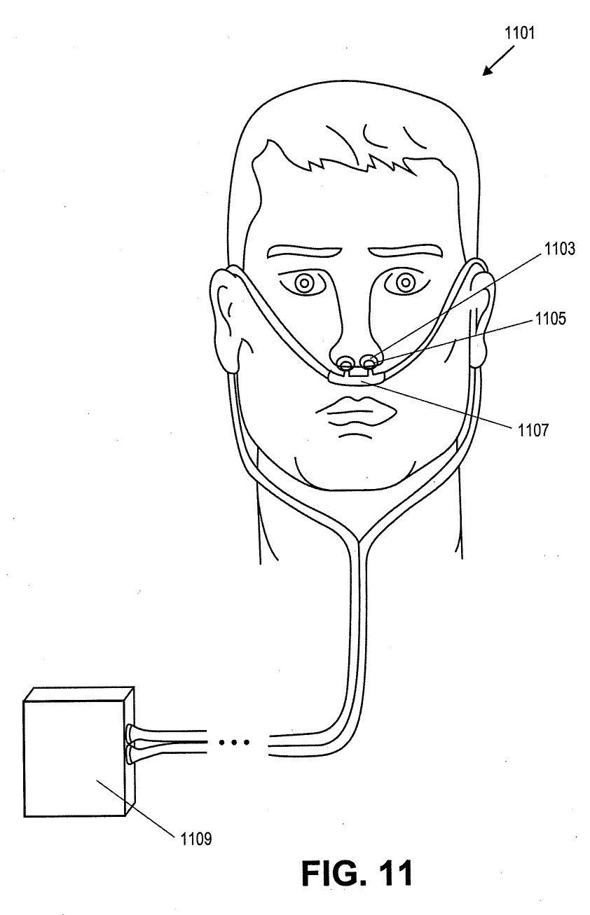

Any of the sensor adapter devices described herein may be included as part of a system for detecting and/or measuring respiration. For example, the devices described herein may be used as part of a system including any of the following components: a nasal respiratory device or devices, a sensor or sensors (including a cannula), and a data acquisition device including a memory or transmitter. FIG. 11 illustrates one variation of a system including all of these elements.

In this variation, the system (shown worn by a subject 1101) includes a pair of adhesive nasal devices 1103. Each nasal device is attached to a sensor adapter 1105, and both sensor adapters are connected to the sensor cannula 1107 which extends from the subject to a sensor unit 1109. The sensor unit may include additional sensor components, and may also include hardware, software and/or firmware for measuring, and storing and/or transmitting information about from the sensor. For example, the sensor attached to the sensor connector may be part of a pressure sensor which reads respiratory pressure from the nasal device through the cannula. In this example, a portion of the pressure sensor (excluding the cannula) may be housed within the sensor unit 1109. Alternatively, the transducer may be attached more proximally to the patient, e.g., near the sensor connector.

Other systems may include different or additional sensors. For example, a sensor for detecting respiration through the subject's mouth may also be included. In some variations, the sensor is not a pressure sensor, but is a temperature sensor (e.g., a thermister, thermocouple, infrared device, etc.). Temperature sensors and pressure sensors may be used in determining a polysomnogram.

Nasal Respiratory Devices with Integral Sensor Connectors

In some variations a separate adapter is not necessary, because the nasal respiratory device includes a sensor connector. In general, the sensor connector is located on the external face (e.g., the distal end) of the nasal device. Nasal respiratory devices including an integral sensor connector typically include a holdfast, one or more passageways through the nasal device, and an airflow resistor configured to increase the resistance to expiration more than the resistance to inspiration, as well as a sensor connector. In general, any nasal respiratory device, particularly those described above, and incorporated by reference, may include an integral sensor connector for connecting and positioning a sensor.

In some variations, the nasal respiratory device includes at least one leak pathway, which may be referred to as an expiratory opening. The leak pathway allows the passage of air through the device during expiration, even when the airflow resistor is closed, and during inspiration. In some variations, the sensor connector is configured so that at least a portion of the sensor detector is aligned (e.g., positioned across from, or within) the leak pathway.

An integral sensor connector may be structured as described above for the sensor connectors that form part of the sensor adapters. Just like the sensor connector of a sensor adapter, an integral sensor connector generally secures a sensor (or a region of a sensor, such as the sampling or detection region) so that it is positioned in communication with an opening of the nasal respiratory device. The integral sensor connector portion of a nasal respiratory device may be configured to secure any appropriate sensor.

The integral sensor connector of a nasal respiratory device may be a mount that holds the sensor in position. The integral sensor connector may be adjustable, so that the sensor can be positioned relative to the sensor adapter and/or the nasal device. In some variations the sensor can be secured more tightly after it has been adjusted (e.g., by clamping or otherwise activating the integral sensor connector). An integral sensor connector may permanently or releasably secure a sensor to the nasal respiratory device.

An integral sensor connector region of a nasal respiratory device may include one or more structures for holding the sensor in place. For example, the integral sensor connector may be configured as a surface that grips or attaches to the sensor. The integral sensor connector may be an opening, tube or passageway into which a portion of the sensor fits. The integral sensor connector may be a protrusion, tube, or prong onto which the sensor is attached. In some variations the integral sensor connector includes one or more adjustable surfaces that can clamp onto a portion of a sensor (e.g., sampling cannula, sensor housing, sensor lead, etc.).

The integral sensor connector may also be keyed to the sensor. Keying may help orient the sensor with respect to the nasal device. For example, the integral sensor connector of the nasal device may be keyed by having an opening into which the sensor inserts that is notched or flattened on one side in compliment with a projection, groove or surface of the sensor.

The integral sensor connector may secure the sensor in any appropriate fashion. For example, the integral sensor connector may be sized to friction fit to the sensor. The integral sensor connector may include an adhesive surface for securing the sensor. The integral sensor connector may include a compressible or clamping surface for securing the sensor. The integral sensor connector surface (or some other portion of the nasal respiratory device) may interlock with the sensor (or a complimentary portion of the sensor). The integral sensor connector may magnetically secure the sensor within the sensor connector. The integral sensor connector may secure a sensor therein using an elastomeric material. For example, the integral sensor connector may contract around or over a portion of the sensor.

An integral sensor connector may be formed as part of a nasal respiratory device. For example, the integral sensor connector may be formed as part of the rim body region forming the passageway through the nasal device. In particular, the integral sensor connector may be formed as part of the distal side which faces outward from the subject when the device is worn. In some variations, the sensor connector is formed as part of the holdfast. For example, the nasal device may include an adhesive holdfast and/or a conformable or compressible holdfast.

In some variations, the integral sensor connector is formed as part of the airflow resistor. For example, the airflow resistor may include a valve (e.g., flap valve) limiter or a crossbeam/cross-strut. The integral sensor connector may be formed as a portion of this.

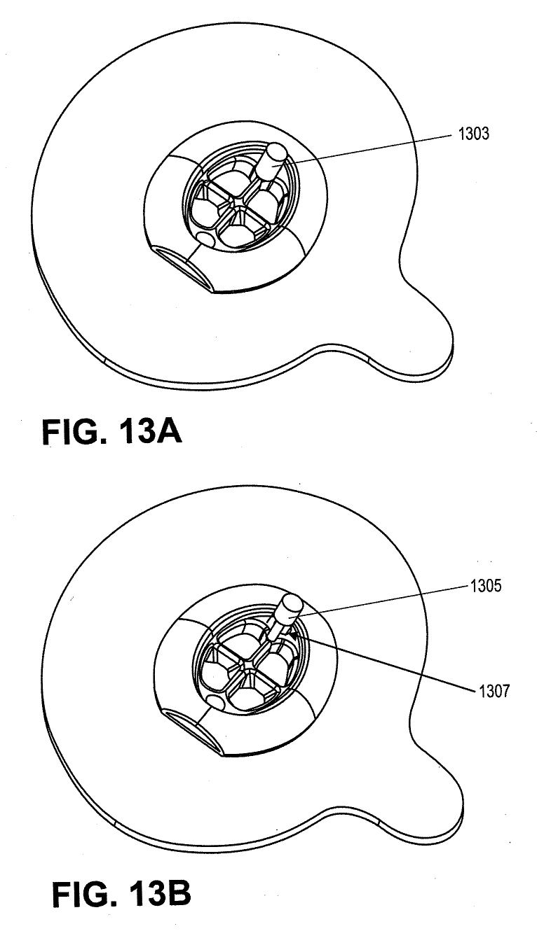

FIGS. 13A and 13B illustrate examples of nasal respiratory devices including an integral sensor. The nasal respiratory device shown in FIG. 13A is very similar to that shown in FIG. 1A, but includes an integral sensor connector 1303. In practice, the sensor connector may be located anywhere on the nasal respiratory device, particularly over an expiratory opening, as shown in FIG. 13A. In some variations there is a window or opening 1307 between the expiratory and/or inspiratory opening from the passageway through the nasal device and the integral sensor connector 1305, as shown in FIG. 13B.

In some variations of the nasal respiratory devices described herein, a sensor (or sensors) is attached to the nasal respiratory device. For example, a sensor may be attached (permanently or removably) to the integral sensor connector.

### Methods of Use

In operation, the sensor adapters allow the measurement of one or more respiratory parameters, particularly when a nasal respiratory device is worn. For example, a sensor adapter may be used to monitor treatment of a sleep disorder when a subject is wearing a nasal respiratory device.

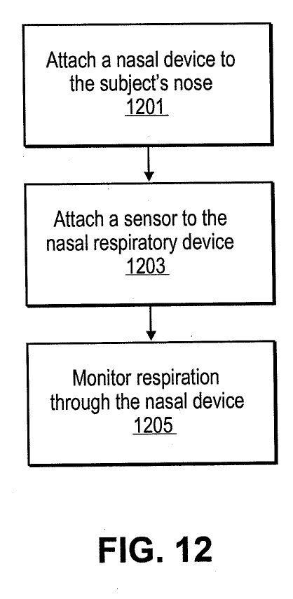

FIG. 12 shows a flowchart illustrating one method of monitoring a treatment of a sleep disorder. According to this example, a nasal device is attached to a subject's nose 1201 in communication with the subject's nasal cavity. Generally, the nasal respiratory device is attached to the subject's nose without interfering with respiration through the subject's mouth. The nasal respiratory device may inhibit expiration more than inspiration. For example, the nasal respiratory device may be a passive nasal respiratory device that include an airflow resistor such as a flap valve, and may also include one or more leak pathways. In some variations the nasal respiratory device is an adhesive device that is adhesively secured to the subject's nose.

A sensor may then be attached to the nasal respiratory device. The sensor may be any appropriate sensor 1203. The sensor may be attached using any of the sensor adapters described herein. Once the sensor is attached, the position of the sensor detector may be adjusted. For example, the sensor detector may be adjusted so that it is positioned opposite of an expiratory opening (e.g., leak pathway) or so that it is opposite both an expiratory and an inspiration-only opening. In some variations, the method also includes the step of locking the sensor in position (e.g., by clamping the sensor connector region). In some variations, the position of the sensor may be adjusted so that it is positioned within an outlet (e.g., expiratory outlet) of the nasal device.

The sensor may be attached permanently or removably to the sensor adapter and the sensor adapter may be attached permanently or removably to the nasal device. For example, the sensor adapter may be permanently or semi-permanently attached to the nasal device by an adhesive that chemically bonds the sensor adapter to the nasal device. The sensor may be either permanently (by adhesive) or removably (e.g., by friction fit) secured by the sensor adapter. Any of the methods of securing either the sensor adapter to the nasal device or the sensor to the sensor adapter may be used.

Once the sensor is attached, respiratory many be monitored by the sensor 1305. Data may be collected for any desired time period, particularly when the subject is sleeping. For example, the sensor may be used to record a polysomnogram. Methods of recording an analyzing polysomnograms may be found, for example, in “Nasal Pressure Airflow Measurement: An Introduction,” by D. Rapoport, et. al. (Pro-tech services, Inc., Mukilteo, Wash., 2001).

As described above, any appropriate sensor may be used. For example, the sensor may allow monitoring of: airflow, air pressure, temperature, humidity, chemical composition, or the like. One exemplary sensor is an air pressure sensor including a cannula. Pressure is measured, and the pressure data may be analyzed to estimate airflow through the nasal device. Thus, the systems described herein may be used to measure airflow through a nasal device (and therefore through the nose). The systems described herein may also be used for measuring air pressure. When a thermister or thermocouple sensor is used, the temperature change due to respiration through the nasal device may be measured. The change in temperature may also be used to determine an estimation of airflow through the device or nose. Similarly, infrared sensors may also be used to measure temperature change and/or flow.

In general, the order in which the steps above are preformed may be different. For example, the sensor may be attached (and adjusted) to the nasal device before it is applied to the subject. The methods described above may be used to monitor treatment of a sleep disorder, or simply to monitor respiration generally. Other treatments or diagnoses, particularly those involving the use of a nasal respiratory device, may also be performed using the devices and systems described herein. Furthermore, although the sensor adapters described above are described for use with a passive nasal devices (e.g., having an airflow resistor configured to inhibit expiration more than inspiration), they may also be used with other nasal device, particularly nasal devices that attach to the nose and include an opening or passageway thorough the body of the nasal device.

While the devices, systems, and methods for using them have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

What is claimed is:

1. A method of monitoring treatment of a sleep disorder comprising:
securing a nasal respiratory device in communication with the subject's nasal cavity without covering the subject's mouth, wherein the respiratory device includes a passive airflow resistor configured to inhibit expiration more than inspiration;
attaching a sensor to the nasal respiratory device; and
monitoring respiration using the sensor.

2. The method of claim 1 further comprising attaching a sensor adapter to the nasal respiratory device.

3. The method of claim 1 further comprising attaching a sensor to an integral sensor connector on the nasal respiratory device.

4. The method of claim 1 further comprising attaching a sensor adapter to the nasal respiratory device so that the resistance to inspiration and to expiration through the nasal respiratory device is not substantially altered.

5. The method of claim 1, wherein the attaching step comprises attaching the sensor to the nasal respiratory device in communication with an outlet of the nasal respiratory device.

6. The method of claim 1, wherein the attaching step comprises positioning a sensor detector input for the sensor in communication with both an expiratory outlet and a valved outlet of the nasal respiratory device.

7. The method of claim 1 further comprising monitoring airflow through the nasal respiratory device.

8. The method of claim 1 further comprising monitoring air pressure from airflow through the nasal respiratory device.

9. The method of claim 1 further comprising monitoring a temperature change from airflow through the respiratory device.

10. The method of claim 1, further wherein the attaching step comprises attaching the sensor to a sensor adapter.

11. The method of claim 1, wherein the step of attaching a sensor to the nasal respiratory device comprises attaching a thermister or thermocouple.

12. The method of claim 1, wherein the step of attaching a sensor to the nasal respiratory device comprises attaching a strain gauge.

13. The method of claim 1, wherein the step of attaching a sensor to the nasal respiratory device comprises attaching a pressure transducer in communication with the nasal respiratory device.

14. The method of claim 1, wherein the step of attaching a sensor to the nasal respiratory device comprises attaching a cannula in communication with a pressure transducer to the nasal respiratory device.

15. The method of claim 1, wherein the nasal respiratory device is adhesively secured in communication with the nasal cavity.

16. The method of claim 1, wherein the step of attaching a sensor to the nasal respiratory device comprises securing the sensor to the nasal respiratory device using a connector selected from the group consisting of: an adhesive, a snap, a magnet, a friction fit, a press fit, a screw, and a hook-and-loop adhesive.

17. A sensor adapter configured to attach to a passive-resistance nasal respiratory device, the adapter comprising:
a body frame including a sensor connector configured to secure a sensor detector input of a sensor in communication with an opening on the nasal respiratory device; and
an attachment site configured to mate with the passive-resistance nasal respiratory device and secure the sensor adapter thereto.

18. The sensor adapter of claim 17, wherein the sensor connector is configured to position the sensor detector input in communication with an outlet of the nasal respiratory device.

19. The sensor adapter of claim 17, wherein the sensor connector is configured to position the sensor detector input in communication with an expiratory outlet and a valved outlet

20. The sensor adapter of claim 17, wherein the sensor connector comprises a channel configured to seat at least a portion of a sensor.

21. The sensor adapter of claim 17, in which the sensor adapter comprises a flange configured to mate to a cannula.

22. The sensor adapter of claim 17, wherein the attachment site is a snap fit connector comprising two or more surfaces configured to secure a portion of a nasal respiratory device.

23. The sensor adapter of claim 17, wherein the attachment site is a press-fit attachment site.

24. The sensor adapter of claim 17, wherein the attachment site comprises an adhesive material.

25. The sensor adapter of claim 17, wherein the attachment site is selected from the group consisting of: a snap, a magnet, a hook-and-latch material, a screw.

26. The sensor adapter of claim 17 further comprising a sensor detector input of a sensor attached to the sensor connector.

27. The sensor adapter of claim 26, wherein the sensor is selected from the group consisting of:
pressure transducer, strain gauge, thermister, thermocouple, and IR sensor.

28. A system for monitoring treatment of a sleep disorder comprising:
a passive-resistance nasal respiratory device including an airflow resistor configured to inhibit expiration more than inspiration;
a sensor adapter configured to secure a sensor in communication with an outlet of the nasal respiratory device, wherein the sensor adapter includes a sensor connector configured to secure at least a portion of a sensor detector input of a sensor in communication with an opening on the nasal respiratory device.

29. The system of claim 28 further comprising a sensor having a sensor detector input connected to the sensor connector and configured to monitor respiration through the nasal respiratory device.

30. The system of claim 28, wherein the sensor is selected from the group consisting of: a pressure sensor, a thermocouple, a thermister, an IR sensor, and a strain gauge.

31. The system of claim 28 further comprising a nasal cannula configured to attach to the sensor connector.

32. The system of claim 28, wherein the sensor connector of the sensor adapter is configured to secure the sensor detector input in communication with an expiratory outlet and a valved outlet of the nasal respiratory device.

33. A sensor adapter configured to attach a nasal cannula to a nasal respiratory device, the adapter comprising a body frame including:
a sensor connector having a surface to which a cannula may be secured so that a distal cannula opening is held in communication with an outlet of the nasal respiratory device; and
an attachment site configured to mate with the nasal respiratory device to secure the sensor adapter to the nasal respiratory device.

34. The sensor adapter of claim 33, further wherein the surface of the sensor connector comprises a surface against which the inner diameter of a cannula may be friction fit to hold the position of the cannula.

35. The sensor adapter of claim 33, wherein the surface of the sensor connector comprises a surface against which the outer diameter of the cannula may be friction fit to hold the position of the cannula.

36. The sensor adapter of claim 33, wherein the cannula is secured to the sensor connector so that the distal cannula opening is held in communication with an expiratory and a valved outlet of the nasal respiratory device.

37. A nasal respiratory device configured to be secured in communication with a subject's nasal cavity, the device comprising:
a passageway configured to communicate with the nasal cavity;
an airflow resistor in communication with the passageway, wherein the airflow resistor is configured to increase the resistance to air exhaled through the passageway more than the resistance to air inhaled through the passageway;
a sensor connector configured to secure a sensor detector input of a sensor in communication with an opening through the device; and
an adhesive holdfast configured to secure the respiratory device in communication with the nasal cavity without covering the subject's mouth at least partly within and/or at least partially over the subject's nasal cavity.

38. The device of claim 37, further wherein the sensor is configured to secure a sensor detector input in communication with a leak pathway and a valved opening through the device.

39. The device of claim 37 further comprising a sensor detector input of a sensor connected to the sensor connector.

40. The device of claim 39, wherein the sensor is selected from the group consisting of: a pressure sensor, a thermocouple, a thermister, an IR sensor, and a strain gauge.

41. A nasal respiratory device adapted to be secured in communication with a subject's nasal cavity comprising:
a passageway configured to communicate with the nasal cavity;
an airflow resistor in communication with the passageway, wherein the airflow resistor is configured to increase the resistance to air exhaled through the passageway more than the resistance to air inhaled through the passageway;
a sensor connector configured to secure a sensor detector input of a sensor in communication with an opening through the device; and
a compressible holdfast configured to secure the respiratory device at least partially within the subject's nasal cavity.

42. The device of claim 41, further wherein the sensor is configured to secure a sensor detector input in communication with a leak pathway and a valved opening through the device.

43. The device of claim 41 further comprising a sensor detector input of a sensor connected to the sensor connector.

44. The device of claim 43, wherein the sensor is selected from the group consisting of: a pressure sensor, a thermocouple, a thermister, an IR sensor, and a strain gauge.
